(19) United States
(12) Patent Application Publication
(10) Pub. No.: US 2009/0098202 A1
(43) Pub. Date: Apr. 16, 2009

(54) Extended Release Formulation

(75) Inventors: Thomas Friedl, Ochsenhausen (DE); Wolfram Eisenreich, Ulm (DE); Sebastian Haertter, Warthausen (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim (DE)

(21) Appl. No.: 12/278,848

(22) PCT Filed: Feb. 9, 2007
(86) PCT No.: PCT/EP07/51258

§ 371 Date: Sep. 16, 2008

(30) Foreign Application Priority Data
Feb. 10, 2006 (EP) 06002777.8

(51) Int. Cl. A61K 9/22; A61K 31/428; A61P 25/00

(52) U.S. Cl. 424/468; 514/367

(57) ABSTRACT

The invention is directed to an extended release formulation comprising pramipexole or a pharmaceutically acceptable salt thereof.

### FIELD OF THE INVENTION

The present invention is directed to an extended release formulation with a selected release profile containing pramipexole or a pharmaceutically acceptable salt thereof, a method for manufacturing the same and use thereof.

### BACKGROUND OF THE INVENTION

Pramipexole is a known dopamine D2 receptor agonist and as such a useful pharmacologically active substance for the treatment of diseases related to the central nervous system (CNS). It is structurally different from the ergot-derived drugs, e.g. bromocriptine or pergolide. It is also pharmacologically unique in that it is a full agonist and has receptor selectivity for the dopamine D2 family of dopamine receptors.

Pramipexole is designated chemically as (S)-2-Amino-4,5,6,7-tetrahydro-6-(propylamino)benzothiazole and has the molecular formula C₁₀H₁₇N₃S and a relative molecular mass of 211.33. The chemical formula is as follows:

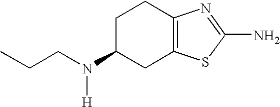

The salt form commonly used is pramipexole dihydrochloride monohydrate (molecular formula C₁₀H₂₁Cl₂N₃OS; relative molecular mass 302.27). Pramipexole dihydrochloride monohydrate is a white to off-white, tasteless, crystalline powder. Melting occurs in the range of 296° C. to 301° C., with decomposition. Pramipexole is a chiral compound with one chiral centre. Pure (S)-enantiomer is obtained from the synthetic process by chiral recrystallization of one of the intermediates during synthesis.

Pramipexole dihydrochloride monohydrate is a highly soluble compound. Water solubility is more than 20 mg/ml and solubility in buffer media is generally above 10 mg/ml between pH 2 and pH 7.4. Pramipexole dihydrochloride monohydrate is not hygroscopic, and of highly crystalline nature. Under milling the crystal modification (monohydrate) does not change. Pramipexole is very stable in the solid state, yet in solution it is light sensitive.

Pramipexole currently is available in form of immediate release tablets (IR tablets), which are used for the treatment of early parkinson's disease or advanced parkinson's disease in combination with levodopa. The IR tablets have to be taken 3 times a day.

From the pharmacokinetic point of view pramipexole IR tablets are rapidly and completely absorbed following oral administration. The absolute bioavailability is greater than 90% and the maximum plasma concentration occurs within 1 to 3 hours. The rate of absorption is reduced by food intake but not the overall extent of absorption. Pramipexole shows linear kinetics and a relatively small inter-patient variation of plasma levels. The elimination half-life (t_{1/2}[h]) varies from 8 hours in the young to 12 hours in the elderly.

As a CNS-active drug substance it is desirable to have an as simple as possible therapy regimen for Pramipexole in order to improve the compliance of the drug. This is beneficial as patients who are suffering from a CNS-related disease often are in a mental state and/or have evolved motor syndromes which makes it difficult to take a long-term treatment drug several times a day. Accordingly, the present extended release formulation allows to simplify the patient's administration scheme by reducing the amount of recommended daily intakes, improves patient's compliance, and attenuates adverse events, e.g. related to high plasma peaks.

Although modified release systems are known in the art, it has proved difficult to formulate a pramipexole tablet having a suitable combination of modified, extended or sustained-release and handling properties, as pramipexole (pramipexole dihydrochloride monohydrate respectively) has a relatively high solubility.

There are a number of approaches described in prior art to provide sustained release tablet compositions of pramipexole:

WO 2004/010997 describes a sustained-release pharmaceutical composition in a form of an orally deliverable tablet comprising a water-soluble salt of pramipexole, dispersed in a matrix comprising a hydrophilic polymer and a starch having a tensile strength of at least about 0.15 kN cm⁻², at a solid fraction representative of the tablet. The disclosure thereof is concentrated to provide a composition with sufficient hardness yield during a high-speed tableting operation.

WO 2004/010999 discloses an orally deliverable pharmaceutical composition comprising a therapeutically effective amount of pramipexole or a pharmaceutically acceptable salt thereof and at least one pharmaceutically acceptable excipient, said composition exhibiting at least one of (a) an in vitro release profile wherein on average no more than about 20% of the pramipexole is dissolved within 2 hours after placement of the composition in a standard dissolution test; and (b) an in vivo pramipexole absorption profile following single dose oral administration to healthy adult humans wherein the time to reach a mean of 20% absorption is greater than about 2 hours and/or the time to reach a mean of 40% absorption is greater than about 4 hours.

It is an object of the present invention to provide a controlled release composition of pramipexole or a pharmaceutically acceptable salt thereof that is suitable for once-daily oral administration.

It is a further object to provide a composition comprising pramipexole or a pharmaceutically acceptable salt thereof that continuously provides a constant plasma level of the active ingredient over the formulations's passage through the gastro-intestinal tract.

### SUMMARY OF THE INVENTION

It has been found that an extended release composition which releases pramipexole constantly within the gastrointestinal tract over a period of at least 4 hours, preferably 8 hours has substantial advantages over other formulations in that it is capable of achieving and maintaining steady state plasma concentrations of the drug which are effective and tolerable.

### DESCRIPTION OF THE INVENTION

The present invention relates to an extended release formulation of pramipexole, one of its pharmacologically acceptable salts respectively, as active ingredient which provides certain pharmacodynamic properties.

For the sake of clarity, in the context of the present description the terms pramipexole, pharmacologically acceptable salt thereof just like pramipexole dihydrochloride or the monohydrate thereof are used interchangeably, while in any context pramipexole dihydrochloride, the monohydrate thereof respectively, is preferred.

Although the formulation according to the invention preferably is a tablet or a capsule comprising the formulation according to the invention, other dosage form designs such as a hard capsule filled with pellets or granules may also be used. Accordingly, it will be appreciated by the skilled person in the art, that excipients that are disclosed in context with a tablet or capsule also may be used for other kind of formulations as well.

The formulation according to the present invention releases the active ingredient in total over a period of at least 4 hours, preferably 8 hours, preferably of at least 12 hours, more preferably of at least 18 hours and even more preferred of at least 24 hours. In particular preferred is a release profile of between 12 and 24 hours. As used herein, the total incorporated dose is defined as from about 90 to about 105% of the incorporated amount of active ingredient according to a suitable assay. The release profile is determined by in vitro dissolution testing according to United States Pharmacopeia (USP) 28, in particular chapter 711, which is incorporated by reference.

Preferably, the time to peak plasma concentrations (tₘₐₓ) of pramipexole according to the present invention is at least about 2.5 hours after administration to a human in the fasted state, preferably 3 hours. Yet further embodiments are characterised by tₘₐₓ values of at least about 3 hours, preferably by tₘₐₓ values being in the range from about 3 to about 12 hours, respectively. Examples of suitable tₘₐₓ values include approx. 4 hours (±0.5 hour), approx. 5 hours (±0.5 hour), and approx. 6 hours (±0.5 hour). As used herein, the tₘₐₓ should be understood as the mean or as used further in this description the median time to peak plasma concentrations, instead of an individual tₘₐₓ determined with only one human subject. In order to determine the median time to peak plasma concentrations (tₘₐₓ) of pramipexole after administration, at least 6 human subjects receiving the respective composition should be involved.

Moreover, the median tₘₐₓ achieved by the composition of the invention is preferably substantially longer than the median tₘₐₓ achieved by the administration of an oral immediate release dosage form comprising pramipexole dihydrochloride monohydrate. In one of the preferred embodiments the median tₘₐₓ of the composition of the invention is at least about 1.5 hours, preferably at least 2 hours and more preferably at least 4 hours and in particular preferably at least 5 hours longer compared to the one of an oral immediate release dosage form. These features apply to the administration in the fasted state, and to median values obtained from several human individuals, as outlined above.

For the sake of clarity, if reference is taken to an oral immediate release dosage form comprising pramipexole dichloride monohydrate, this dosage forms is a tablet comprising pramipexole dichloride monohydrate in an amount of 0.125 mg or 0.25 mg or 0.5 mg or 1 mg, mannitol, corn starch (maize starch), colloidal silicium dioxide, povidone, magnesium stearate. This tablet is available under the brand name Sifrol® in Germany. A typical immediate release tablet in Germany is known under the trade name Sifrol®

Preferably, the release behaviour of the composition of the invention is robust with regard to whether the individual receiving the medication is in the fed or fasted state. The robustness of the composition may be expressed in terms of the absolute difference between the respective median tₘₐₓ values after administration in the fed and fasted states. For example, the difference should preferably be less than about 4 hours. More preferably, the difference in median tₘₐₓ between the fed and fasted state should be less than about 3, preferably less than about 2.5 hours. Particularly if the release profile of the composition is selected to achieve a tₘₐₓ in the fasted state of about 2.5 to about 6 hours, preferably 2.5 to about 4 hours the tₘₐₓ in the fed state should preferably not be different by more than about 3 hours, preferably more than about 2 hours. The maximum concentration and the area under concentration-time curve (AUC) after administration in the fed state should not exceed a difference of ±30%, preferably ±20% compared with the fasted state.

In another embodiment of the present invention, an oral extended release composition is provided which releases pramipexole according to a release profile which may be adapted to achieve a peak-through-fluctuation (PTF) of pramipexole plasma concentrations upon once daily administration after reaching steady state of less than about 100%. Preferably, the PTF may be not higher than that which is obtained when an immediate release formulation of the same active ingredient is administered three times a day to fasted humans after reaching steady state.

According to another aspect, the invention provides oral extended release compositions comprising pramipexole or one of its pharmaceutically acceptable salts, derivatives, solvates, and isomers, wherein the release profile is adapted to achieve and/or sustain average pramipexole plasma concentrations (C_{avg}), preferably over a 12 h period, more preferably over a 15 hour period, more preferably over a 18 h period and most preferably over a 24 h period which are at the same range as will be reached at steady state after applying the same total daily dose of the immediate release formulation. For example for a daily dose of 0.75 mg the geometric mean of C_{avg} should be between 0.3-1.2 ng/mL ideally it should be between 0.5-0.8 ng/mL, preferably 0.6-0.8 ng/nL.

Similarly, it is preferred that the average plasma concentration of pramipexole upon administration in the fasted state after reaching steady state does not differ by more than 25% from the average plasma concentration of pramipexole upon administration of an immediate release formulation thrice daily of the same active ingredient at the same daily dose in the fasted state after reaching steady state. For the sake of comparability, the percentage should be calculated as relative to average plasma concentration reached after thrice daily dosing of the immediate release formulation. According to another preferred embodiment, the respective average plasma concentrations do not differ by more than about 20%.

As used herein, reaching steady state means that a regular with respect to time dosing regimen has been followed for a sufficiently long period of time so that the average plasma concentration of the active compound after administration is similar to the average plasma concentration after the previous administration. Similarly, the trough plasma concentration is similar to the respective concentrations after the previous dosing.

For the avoidance of misunderstandings, average plasma concentrations (C_{avg}) does not mean that the trough or minimum plasma concentrations at steady state should be within the specified ranges: Moreover, the average plasma concentrations refer to the means of average plasma concentrations at steady state determined for at least 6 individuals.

Although the mean peak and trough concentrations may be outside the ranges preferred for the average plasma concentrations, the fluctuations between these extreme values at steady state should be moderate.

In one embodiment the release profile of the composition of the invention may be adapted to achieve a PTF of less than about 1 (=100%) after reaching steady state conditions with the IR-formulation given thrice a day. In another preferred embodiment, the peak-to-trough fluctuation may be less than about 90%. If further embodiments, the release profile of the composition is adapted to result in a peak-to-trough fluctuation of less than about 85%, or a peak-to-trough fluctuation of less than about 80%, respectively. In yet a further embodiment, the peak-to-trough fluctuation may be approximately 75% or less. Preferably, these moderate fluctuations are achieved in a once-daily administration regimen.

Again, it should be noted that, according to the present invention, all plasma concentrations and all parameters like C_{avg} and PTF derived therefrom are mean values obtained from a group of at least 6 individuals.

The composition shall upon once daily dosing and after reaching steady state conditions, result in pramipexole peak plasma concentrations which are not much different from the peak plasma concentrations obtained from the known thrice daily administration of an immediate release formulation containing a third of the dose of the same active ingredient. More precisely, it is preferred that the composition of the invention leads to steady state peak plasma concentrations which are lower than the steady state peak plasma concentrations of such immediate release formulation.

If, for example, pramipexole dihydrochloride monohydrate is selected as active ingredient to perform the invention, and the amount of active ingredient incorporated in a dosage unit is 0.75 mg, a suitable immediate release formulation is the commercially available Mirapex Tablet with the strength of 0.25 mg. Administered once a day, the composition leads to steady state peak plasma concentrations which are lower than the steady state peak plasma concentrations of this Mirapex Tablet given thrice daily. In further embodiments, the steady state peak plasma concentration shall not be lower than about 15% or 10% compared to the steady state peak plasma concentration of the immediate release formulation given three times a day.

As mentioned above, three times a day includes dosing regimens in which the time intervals between the first and the second administration, or between the second and the third administration, do not have to be of the same length as the time interval between the third administration on one day and the first administration on the following day. For example, regular administration at about 7 a.m., 1 p.m. and 7 p.m. is within the scope of a thrice daily regimen.

Not only the peak plasma concentrations, but also the trough plasma concentrations of pramipexole at steady state are similar between the composition of the invention when administered once daily and an immediate release formulation containing a third of the dose of the same active ingredient given thrice daily. Preferably, the morning trough plasma concentration, immediately before the first morning dose, of pramipexole upon administration in the fasted state after reaching steady state is equal or higher than the morning trough plasma concentration of pramipexole upon administration of an immediate release formulation comprising a third of the dose of the same active ingredient in the fasted state after reaching steady state. In other embodiments, the trough plasma concentration of the composition is not more than about 20%, or not more than 15%, or not more than 10%, above that achieved by the immediate release formulation administered thrice daily, respectively.

Furthermore, the inter-individual variability in total daily exposure (reflected by the area under the concentration-time curve from time 0 to 24 h) does not exceed the inter-individual variability of the immediate release formulation, given at the same daily dose.

In a preferred embodiment of the present invention, the extended release composition releases the incorporated active ingredient substantially independent of the pH of the dissolution medium, at least in a defined pH range below 8. A substantially pH-independent release profile means that the release profile, when determined in the same model, apparatus, under comparable conditions (such as using the same volume of dissolution medium) and with the same apparatus settings (such as the same rotation speed), the release profile of a composition in a dissolution medium having a first pH is similar to the release profile of the same composition in a dissolution medium having a second pH, wherein the first and the second pH are different from each other, but both within the physiologically relevant range of below about pH 8, preferably of between 1 and smaller 8. Preferably, the drug release occurs substantially independent of the pH of the dissolution medium, as long as the pH is selected within the physiological range.

In particular, the respective release profiles have a similar overall shape, and the (dose independent) released fractions of active ingredient at any point of time do not differ between the release profiles by more than about 20% relative to the incorporated dose of the active ingredient. More preferably, the released fractions of active ingredient at the majority of points of time do not differ by more than about 15% relative to the incorporated dose. In further embodiments, the difference at any point of time is not more than about 15%, or not more than about 10%, or not more than about 7.5%, respectively, relative to the incorporated dose. As understood herein, a release profile is determined by testing at least three samples of a particular composition and calculating averages of the released amounts of active ingredient at each point of time.

It has been found that an extended release composition which releases pramipexole in a pH-independent manner over the specified period of time has substantial advantages over other formulations in that it is capable of achieving and maintaining steady state plasma concentrations of the drug which are effective and tolerable, and which are at low risk of any unexpected release.

According to another embodiment of the invention, the release profile is not only substantially independent of the pH of the dissolution medium, but also substantially constant over the whole gastrointestinal tract including colon.

In one of the preferred embodiments, the pH of the dissolution medium is selected in the range of about 1.2 to about 7.3, preferably from about 1.2 to about 6.8. Alternatively, the pH is selected in the range from about 3 to about 7.3, preferably from about 4.5 to about 6.8. Depending on the intended use of the composition any of these pH ranges may be considered appropriate or relevant for determining potential pH-effects on drug release including the absence of a pH-effect.

In another embodiment the release profile may show limited, substantially pH-independency within a range of pH from 4.5 to 7.5, whereas it is not in a pH range of 1 to <4.5. In such embodiments the release within the range of pH of 1 to smaller 4.5 may be faster than in the pH range of 4.5 to 7.5. The ranges may vary within a range of +/−1.5, preferably +/−1, more preferably +/−0.5.

The composition of the invention can be prepared by formulation techniques and from excipients which are generally known to a person trained in the field. Typically, at least one release-sustaining excipient is incorporated into the composition in order to provide a slow release. The release characteristic preferably is pH-independent or at least substantially independent in a pH range of 4.5 to 7.5. Such release-sustaining excipient may, for example, be selected from the group consisting of pharmaceutically acceptable polymers, lipids and waxes.

Polymers are natural or synthetic compounds or mixtures of compounds formed by the polymerisation of small, monomeric compounds and consisting essentially of repeating structural units derived from the monomeric compounds. Examples of pharmaceutically acceptable polymers which are known to have a potential for effecting a slow release of active ingredients from pharmaceutical compositions include members of the following categories:

Polysaccharides, such as cellulose and cellulose derivatives, including alkyl-, hydroxyalkyl- and hydroxyalkyl alkyl cellulose, methylcellulose, hydroxymethylcellulose, ethylcellulose, hydroxyethylcellulose, hydroxypropylcellulose, hydroxybutylcellulose, hydroxyethyl methylcellulose, hydroxypropyl methylcellulose, hydroxypropyl methylcellulose phthalate, carboxymethylcellulose, carboxymethylethylcellulose, cellulose acetate, cellulose acetate butyrate, cellulose acetate propionate, acacia, agar, alginic acid, amylose, amylopectin, carrageenan, chitin, chitosan, trimethylchitosan, galactomannans, guar gum, inulin, locust bean gum, pectin, carboxymethylamylopectin, starch, hyaluronic acid, hydroxyethylstarch, tragacanth, polyfructans, xanthan gum, including any salts and derivatives thereof, in particular the sodium, potassium, and calcium salts of the anionic polymers, and chemically or physically crosslinked forms of any of these polymers;

proteins, such as albumin, casein, collagen, elastin, gelatin, soy protein, whey protein, zein, including the respective salts, derivatives, and crosslinked forms;

other natural polymers such as shellac;

synthetic polymers, such as polyacrylic acid, polyacrylates, polymethacrylic acid, polymethacrylates, acrylic and/or methacrylic acid copolymers, ammonio methacrylate copolymers, polyvinylalcohol, polyvinyl acetate, polyvinylpyrrolidone, copolymers of vinylpyrrolidone and vinyl acetate and of polyvinylalcohol and polyvinylpyrrolidone, polyalkylene oxides such as polyethylene oxide, polypropylene oxide, copolymers of ethylene oxide and propylene oxide, polylactide, polyglycolide, polylactide-co-glycolide, polycaprolactone, poly(dioxanone), poly(hydroxybutyrate), poly(malic acid), poly(ortho esters), poly(ethylene vinyl acetate), aliphatic and aromatic polyanhydrides, poly(ether esters), poly(phosphoesters), for example poly(phosphoesters) based on poly(ethylene terephthalate), poly(phosphoesters) based on cyclohexane-1,4-dimethyphosphate, poly(phosphoesters) based on phosphate-extended lactides, including any salts, derivatives, and crosslinked forms.

Among the preferred polymers from those listed above are neutral polymers, anionic polymers, insoluble polysaccharides, gellable polysaccharides, methacrylic acid copolymers, and ammonio methacrylate copolymers.

Examples of such polymers are water swelling substantially neutral polymers or water swelling anionic polymers.

According to the present invention, preferably a water swelling substantially neutral polymers are used. Among such polymers are

alkylcelluloses, such as, methylcellulose; hydroxyalkylcelluloses, for example, hydroxymethylcellulose, hydroxyethylcellulose, hydroxypropylcellulose and hydroxybutylcellulose; hydroxyalkyl alkylcelluloses, such as, hydroxyethyl methylcellulose and hydroxypropyl methylcellulose; carboxyalkylcellulose esters; other natural, semi-synthetic, or synthetic di-, oligo- and polysaccharides such as galactomannans, tragacanth, agar, guar gum, and polyfructans; methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides such as polyethylene oxide and polypropylene oxide and copolymers of ethylene oxide and propylene oxide, preferably cellulose ether derivatives such as hydroxypropyl methylcellulose and hydroxypropyl cellulose, most preferred hydroxypropyl methylcellulose.

As also water swelling anionic polymers are among the preferred adjuvants of the current invention, the following examples for such polymers shall be given: acrylic acid polymerisate, methacrylic acid copolymers, alginates, carrageenans, acacia, xanthan gum, chitin derivates such as chitosan, carmellose sodium, carmellose calcium, preferably acrylic acid polymerisate.

Lipids are a diverse range of compounds, including relatively water-insoluble or nonpolar compounds of biological origin, including waxes, fatty acids, fatty-acid derived phospholipids, sphingolipids, glycolipids and terpenoids, such as retinoids and steroids.

Waxes, as used herein, refer to relatively lipophilic substances which are, like fats, plastic at room temperature and a liquid of low viscosity above the melting point. Traditionally, a wax was chemically defined as an ester of a monohydric long-chain fatty alcohol and a long-chain fatty acid. According to the invention, however, waxes should be defined more broadly, as has become commonly accepted, to include a broad range of chemically heterogeneous materials, such as glycerides, fatty alcohols, fatty acids, and the esters thereof. Hence, the terms “wax”, “lipid”, and “fat” may not always be distinguishable from one another.

Examples of potentially useful lipids and waxes for practising the current invention include:

Glycerides, such as mono-, di-, and triglycerides of short-, medium-, and long-chain fatty acids, in particular of lauric, myristic, palmitic, stearic, arachidic, linolenic, docosahexaenoic, eicosapentaenoic, linoleic, arachidonic, oleic, and erucic acid, as for example, natural and synthetic fats and fatty oils, hydrogenated fats and oils, hydrogenated vegetable oil, hydrogenated castor oil, glyceryl monooleate, glyceryl monostearate, glyceryl palmitostearate, olive oil, peanut oil, sesame oil, soybean oil, etc.;

Fatty acids, such as lauric, myristic, palmitic, stearic, arachidic, linolenic, docosahexaenoic, eicosapentaenoic, linoleic, arachidonic, oleic, and erucic acid;

Fatty alcohols, such as lauric, myristic, palmitic, stearic, arachidic, linolenic, docosahexaenoic, eicosapentaenoic, linoleic, arachidonic, oleic, and erucic alcohol;

Esters of fatty acids with alcohols other than glycerine, such as ethyl oleate, oleyl oleate, myristyl palmitate etc.;

Natural and semisynthetic waxes or mixtures of lipid-like and wax-like compounds, such as beeswax, Chinese wax, spermaceti, cetaceum, lanolin, carnauba wax, ceresin wax, montan wax, ozocerite, bleached wax, petroleum wax, paraffin wax, etc.;

Derivatised glycerides, fatty acids, and fatty alcohols, such as polyoxyethylene-substituted (or polyethoxylated) fatty alcohols (i.e. polyoxyethylene alkyl ethers), triglycerides (e.g. polyoxyethylene castor oil derivatives), fatty acids (e.g. polyoxyethylene stearates), fatty acid esters (e.g. polyoxyethylene sorbitan fatty acid esters), etc.

Besides, the formulation of the present invention may also optionally comprise further excipients, i.e. pharmaceutically acceptable formulating agents, in order to promote the manufacture, compressibility, appearance and taste of the preparation. These formulating agents comprise, for example, diluents or fillers, glidants, binding agents, granulating agents, anti-caking agents, lubricants, flavors, dyes and preservatives. Other conventional excipients known in the art can also be included.

The filler may be selected from soluble fillers, for example, sucrose, lactose, in particular lactose monohydrate, trehalose, maltose, mannitol and sorbitol. Different grades of lactose can be used. In case of a water soluble active ingredient, like the one described in this invention, more preferably water insoluble fillers, such as starch and starch derivates other than pregelatinized starch, e.g. corn starch, potato starch, rice starch or wheat starch, microcrystalline cellulose, dibasic calcium phosphate dihydrate and anhydrous dibasic calcium phosphate, preferably corn starch, can be used in addition or instead of the water soluble fillers. The total weight percentage of filler ranges between about 5% and about 75% by weight.

A glidant can be used to improve powder flow properties prior to and during tableting and to reduce caking. Suitable glidants include colloidal silicon dioxide, magnesium trisilicate, powdered cellulose, talc, tribasic calcium phosphate and the like. Colloidal silicon dioxide is preferably included as a glidant in an amount up to about 2%, preferably about 0.2% to about 0.8%, by weight of the dosage unit.

If the composition of the invention is designed as a tablet, a lubricant can be used to enhance release of a tablet from apparatus on which it is formed, for example by preventing adherence to the face of an upper punch (“picking”) or lower punch (“sticking”). Suitable lubricants include magnesium stearate, calcium stearate, canola oil, glyceryl palmitostearate, hydrogenated vegetable oil, magnesium oxide, mineral oil, poloxamer, polyethylene glycol, polyvinyl alcohol, sodium benzoate, sodium lauryl sulfate, sodium stearyl fumarate, stearic acid, talc, hydrogenated vegetable oil, zinc stearate and the like. In one embodiment, magnesium stearate is included as a lubricant in an amount of about 0.1% to about 1.5%, preferably about 0.3% to about 1%, by weight of the tablet.

Among the optional formulating agents that further may be comprised in the formulation there may be mentioned agents such as polyvidone; copovidone; starch; acacia; gelatin; seaweed derivatives, e.g. alginic acid, sodium and calcium alginate; cellulose, preferably microcrystalline cellulose, cellulose derivatives, e.g. ethylcellulose, hydroxypropylcellulose, hydroxypropyl methylcellulose, having useful dry or wet binding and granulating properties; and antiadherents such as talc and magnesium stearate.

More than one sustained-release excipient may be used to prepare the composition, for example two or more polymeric excipients, two or more lipids and/or waxes, or a combination of one or more lipid or wax with one or more polymer. Whether a certain excipient is a sustained-release excipient in a particular formulation can be determined, for example, by preparing the respective formulation with and without the excipient, optionally replacing the excipient by another one which is assumed to have no release-sustaining effect (e.g. lactose), and comparing the in vitro release profiles of the formulations. A release-sustaining effect may be assumed if the time for 50% of the incorporated active ingredient is increased by more than about 20%. Other methods may also be useful to determine whether an excipient has a release-sustaining effect.

In a preferred embodiment, the formulation comprises pramipexole or a pharmaceutically acceptable salt thereof in a matrix comprising at least one water swelling polymer other than pregelatinized starch.

In another preferred embodiment in which the release of pramipexole is independent from the pH-value, at least two polymers are present in the composition, of which at least one is a substantially neutral polymer other than pregelatinized starch. Examples of such polymers have been mentioned above.

In another preferred embodiment in which the release of pramipexole is dependent from the pH-value at least two polymers are present in the composition, of which at least one is an anionic polymer, preferably other than pregelatinized starch. Examples of such polymers have been mentioned above. Examples for such anionic are given above. Preferably it is selected from the group or optionally crosslinked acrylic acid polymers, methacrylic acid polymers, alginates, and carboxymethylcellulose. In a preferred embodiment of the present invention the anionic polymer is an optionally crosslinked acrylic acid polymer, preferably with a content of the optionally crosslinked acrylic acid polymer in the matrix from about 0.25 wt.-% to about 25 wt.-%, and preferably from about 0.5 wt.-% to about 15 wt.-%, and preferably from about 1 wt.-% to about 10 wt.-%.

The water swelling polymer represents at least one hydrophilic water swelling polymer which may form an extended release matrix which slowly releases the pramipexole or its salt as active ingredient. The polymer swells upon contact with aqueous fluid following administration, resulting in a viscous, drug release regulating gellayer. The viscosity of the polymer preferably ranges from 150 to 100,000 mPa·s (apparent viscosity of a 2% aqueous solution at 20° C.). In such dosage form design, the amount of water swelling polymer may range from about 10 to about 80% by weight.

Examples of suitable matrix-forming hydrophilic water swellable polymers include different viscosity grades of hydroxypropyl cellulose and hydroxypropyl methylcellulose which are commercially available. Hydroxypropyl methylcellulose (HPMC) preferably used in the present invention has a viscosity grade ranging from about 3,500 mPa·s to about 100,000 mPa·s, in particular ranging from about 4,000 mPa·s to about 20,000 mPa·s and most in particular a viscosity grade of about 6,500 mPa·s to about 15,000 mPa·s (apparent viscosity of a 2% aqueous solution at 20° C.), e.g. hypromellose 2208 or 2206 (DOW, Antwerp, Belgium). HPMC type 2208 contains 19-24% by weight methoxy and 4-12% by weight hydroxypropoxy substituents.

Hydroxypropyl cellulose having a viscosity higher than 1,500 mPa·s (apparent viscosity of a 1% aqueous solution at 20° C.) is preferred, in particular hydroxypropyl cellulose having a viscosity in the range from about 1500 to about 3000 mPa·s, preferably from 4000 to 6500 mPa·s (2% aqueous solutions), e.g. the Klucel series such as Klucel M (Hercules, Wilmington, USA).

In addition, when using a combination of polymers, the ratio of said polymers also influences the release profile of the preparation. A combination of different polymers offers the possibility of combining different mechanisms by which pramipexole is released from the matrix. Such combination facilitates control of the pharmacokinetic release profile of the preparation at will. For example, when using one or more water swelling polymers, in particular hydroxypropyl cellulose and hydroxypropyl methylcellulose, the weight percentage of hydroxypropyl methylcellulose preferably ranges from 25 to about 62%; the weight percentage of hydroxypropyl cellulose preferably ranges between 0% and about 16% or alternatively 1% and about 16%.

Without wishing to be bound by theory, the release of pramipexole or a salt thereof from a matrix containing hydroxypropyl cellulose and hydroxypropyl methylcellulose would probably occur by a combined set of release mechanisms. Due to the higher solubility of hydroxypropyl methylcellulose compared with hydroxypropyl cellulose, the former will gradually dissolve and erode from the matrix, whereas the latter will more act as a sponge-like matrix former releasing the active ingredient mainly by diffusion.

According to one of the embodiments of the present invention, the matrix of the extended release tablet or capsule formulation comprises or essentially consists of hydroxypropyl methylcellulose, such as hypromellose, acrylic acid polymeriate (the latter only for systems with pH dependent release profiles) and further excipients. The amount of hydroxypropyl methylcellulose is preferably in the range from 10 to 75%, particularly preferred from 25 to 65% most preferred from 35 to 55% by weight. The amount of further excipients is preferably in the range from 90 to 25%, particularly preferred from 75 to 35%, most preferred from 65 to 45% by weight.

The expression “consisting essentially” is understood in the sense that it does not in principle exclude the presence, in addition to the mandatory components mentioned, of other components, the presence of which does not affect the essential nature of the formulation.

As active ingredient, pramipexole or a pharmaceutically acceptable salt thereof, may be present in any amount suitable for the desired treatment of a patient. A preferred salt of pramipexole is the dihydrochloride salt, most preferably in the form of the monohydrate. Usual amounts are from about 0.1 to about 5 mg pramipexole salt. According to a particularly preferred embodiment e.g. 0.750 mg pramipexole dihydrochloride monohydrate, corresponding to 0.524 mg anhydrous base, is used in the extended release tablet or capsule formulation according to the present invention. However, any other amount of active ingredient suitable for treatment may be used with the only proviso that the amount of pramipexole or salt is sufficient to provide a daily dose in one to a small plurality, for example one to about 4, of tablets to be administered at one time. Preferably the full daily dose is delivered in a single tablet or capsule. An amount of pramipexole salt, expressed as pramipexole dihydrochloride monohydrate equivalent, of about 0.1 to about 10 mg per tablet or capsule, or about 0.05% to about 5% by weight of the composition, will generally be suitable. Preferably an amount of about 0.2 to about 6 mg, more preferably an amount of about 0.3 to about 5 mg, per tablet or capsule is present. Specific dosage amounts per tablet or capsule e.g. include 0.375, 0.5, 0.75, 1.0, 1.5, 3.0 and 4.5 mg pramipexole dihydrochloride monohydrate. The amount that constitutes a therapeutically effective amount varies according to the condition being treated, the severity of said condition, and the patient being treated.

The extended release particles may be designed as particles having an extended release coating or, alternatively, as hydrophobic matrix particles in which the active ingredient is embedded. Suitable coating compositions typically comprise at least one release-sustaining polymer which is insoluble both in gastric and in intestinal fluid, and preferably also a plasticiser. Among the preferred and pharmaceutically acceptable polymers on which such coatings may be based are ethylcellulose, cellulose acetate, cellulose acetate butyrate, insoluble types of methacrylic acid copolymers, such as ethyl acrylate-methyl methacrylate copolymer and ammonio methacrylate copolymers, polyvinyl acetate, and blends of polyvinyl acetate and polyvinylpyrrolidone.

Any plasticisers should be selected with regard to the choice of polymer. Potentially suitable plasticisers include polyethylene glycol, glycerol, propylene glycol, sorbitol, triacetin, diethyl phthalate, triethyl citrate, tributyl citrate, acetyltributyl citrate, acetyltriethyl citrate, dibutyl sebacate, and dibutyl phthalate. The amount of plasticiser needed will also be dependent on whether the particles are shaped as pellets, irregular granules, or mini-tablets, and on whether the particles will be filled into hard capsules or compressed into tablets.

In one embodiment there is no coating present on the tablet formulation according to the present invention. However, the extended release formulation of the invention may comprise a nonfunctional coating. A nonfunctional coating can comprise a polymer component, for example HPMC, optionally with other ingredients, for example one or more plasticizers, colorants, etc. The term “nonfunctional” in the present context means having no substantial effect on release properties of the tablet, and the coating serves another useful purpose. For example, such a coating can impart a distinctive appearance to the tablet, provide protection against attrition during packaging and transportation, improve ease of swallowing, and/or have other benefits. A nonfunctional coating should be applied in an amount sufficient to provide complete coverage of the tablet. Typically an amount of about 1% to about 10%, more typically an amount of about 2% to about 5%, by weight of the tablet as a whole, is suitable.

Tablets according to the present invention can be of any suitable size and shape, for example round, oval, polygonal or pillow-shaped, and optionally bear nonfunctional surface markings. According to the present invention it is preferred that the extended release tablets are white to off-white and of oval or round, biconvex, shape.

Tablets of the invention can be packaged in a container, accompanied by a package insert providing pertinent information such as, for example, dosage and administration information, contraindications, precautions, drug interactions and adverse reactions.

The release-sustaining excipient may be incorporated together with the active ingredient and, optionally, further excipients, within the same compartment of the dosage form of the composition. For example, both the active ingredient and the release-sustaining excipient, or at least one of the release-sustaining excipients if more than one are present in the composition, may be dispersed in the matrix of an extended release matrix tablet. Dispersed, as used herein, refers to the relatively homogeneous distribution of small particles or molecules.

The principles of designing and preparing extended release matrix tablets and similar matrix-based oral dosage forms are generally known to the person trained in the field.

Alternatively, the composition may be designed as a dosage form comprising at least one active ingredient-containing compartment and at least one release-sustaining compartment, so that the active ingredient and the release-sustaining excipient(s) are accommodated in separate and distinct compartments. For example, the active ingredient may be incorporated within the core of a tablet, a hard capsule, or a soft capsule, and the release-sustaining excipient may be comprised in a barrier coating surrounding the core.

For the avoidance of misunderstanding, it should be noted that even in the case of compositions designed to comprise distinct active ingredient-containing and release-sustaining compartments, it may be useful to incorporate one or more release-sustaining excipients also within the active ingredient-containing compartment. Furthermore, it may be useful to incorporate a portion of the active ingredient within the release-sustaining compartment.

In other embodiments, the release-sustaining excipient is incorporated within one or more distinct layers of a layer tablet. If the release-sustaining component which comprises at least one release-sustaining excipient is shaped like a layer, such as the layer of a tablet, this layer is preferably in contact with the active ingredient-containing compartment, which may also be in the form of a layer. Thus, examples of suitable dosage form designs include layer tablets having two, three or more layers, film-coated tablets, dry-coated (i.e. press-coated) tablets, coated hard capsules, soft capsules whose shells have been modified e.g. by crosslinking, bilayer extrudates, etc.

Further examples include coated granules, coated pellets, coated mini-tablets, or other coated small units filled in hard capsules. In these embodiments, a dosage unit of the composition of the invention comprises a plurality of release-sustaining compartments each of which comprises the, or at least one of the, release-sustaining excipient(s), as well as a plurality of active ingredient-containing compartments. Typically, each of the active ingredient-containing compartments is associated with (e.g. covered with) a release-sustaining excipient.

If the release-sustaining component is coating or covering an active ingredient-containing core, the coating is typically designed to resist disintegration during drug release. Preferably, the release-sustaining compartment of the composition of the invention is poorly soluble in aqueous media at 37° C. The active ingredient is typically released by diffusion through the coating, or by diffusion or convective flow of liquid through one or more openings in the coating. The principles of designing and preparing oral dosage forms having a release-sustaining coating as well as of extended-release layer tablets are generally known to the person trained in the field. Guidance may also be obtained in the afore-cited documents.

In a further embodiment, the composition is designed as a solid dosage form which is adapted to disintegrate rapidly in stomach. Upon disintegration, particles are released which contain the active ingredient and which have extended release characteristics. Preferably, the active ingredient-containing particles have an average diameter which allows them to transit the stomach independently of the digestive state, i.e. whether fed or fasted. It is known that small particles exit the stomach at about the same rate as the liquid content of the stomach, whereas larger unit such as non-disintegrating tablets are emptied at different rates depending on the digestive state. In the fed state, the residence time of relatively large units is typically in the range of several hours. In the fasted state, these units may be expelled rapidly because, during this phase, the strongest peristaltic contractions of the muscular stomach wall occur. The design of the dosage form as comprising multiple extended-release particles of small size is therefore a means to further reduce the sensitivity of a composition to some variable physiological effects related to the gastric residence time and the mechanical stress resulting from gastric motor activity.

The dosage form design may be a hard capsule or a tablet. A hard capsule may simply be filled with small multiple units, such as extended release granules, extended release pellets, or extended release mini-tablets. After ingestion and arrival of the capsule in the stomach, it typically disintegrates within a few minutes, preferably in less than about 30 minutes. More preferably, disintegration occurs within about 20 minutes or less, or within 15 minutes or less.

If a compressed tablet is used as dosage form design for this embodiment, it should also be adapted to disintegrate into the smaller functional units within less than 30 minutes in gastric fluid at 37° C. Again, it may also be useful to achieve a disintegration time of 20 minutes or less, or of 15 minutes or less, respectively. Depending on the design of the extended release particles—e.g. pellets, granules, or mini-tablets, care should be taken to preserve the extended release functionality of the particles during compression. For example, if each particle has a polymeric film coating to effect extended release, the coating should not be brittle, but comprise an adequate amount of an appropriate plasticiser in order to withstand compression without being ruptured.

Furthermore, the present invention is also directed to a method of manufacturing the extended release tablet formulations via a direct compression process comprising the steps of:

(1) producing an active ingredient trituration wherein the active ingredient is pramipexole or a pharmaceutically acceptable salt thereof by preblending it with a portion of water swelling polymer(s) and/or further excipient(s) in a mixer, wherein pramipexole or the pharmaceutically acceptable salt thereof is milled, preferably peg-milled, prior to use;
(2) premixing the active ingredient trituration of step (1), the main portion of the water swelling polymer(s) and/or excipients in a mixer to obtain a pre-mixture;
(3) optionally dry screening the pre-mixture through a screen in order to segregate cohesive particles and to improve content uniformity;
(4) mixing the pre-mixture of step (2) or (3) in a mixer, optionally by adding remaining excipients to the mixture and continuing mixing; and
(5) tableting the final mixture by compressing it on a suitable tablet press to produce matrix tablets.

The trituration step may be omitted, in this case the components of the formulation as described in step 1 may be premixed with the remaining components of step 2 without prior trituration.

To achieve adequate content uniformity in this low drug load formulation, the active ingredient is preferably peg-milled. Preferably the particle size distribution of the peg-milled drug substance, as determined by laser diffractometry using a dry dispensing system, is characterized by particle fraction of 90% (V/V) being smaller than 100 micrometer, more preferably a particle fraction of 90% (V/V) being smaller than 90 μm, most preferably a particle fraction of 90% (V/V) being smaller than 75μ micrometer in diameter.

Also other processes can be applied to the manufacturing of Pramipexole extended release tablets, like conventional wet granulation and roller compaction. In case of wet granulation preferably Pramipexole is granulated with suitable fillers, like e.g. starches other than pregelatinized starch, microcrystalline cellulose, lactose monohydrate or anhydrous dibasic calcium phosphate, and wet binding agents, like e.g. hydroxypropylmethylcellulose, hydroxypropylcellulose, povidone, copovidone, and starch paste, leading to a active ingredient concentrate, which after drying and dry screening is mixed with the main fraction of gel forming excipients, like all the above described retarding principles.

In case of roller compaction, or in other words dry granulation, either a premix of Pramipexole with part of the excipients used in the direct compression process, or the complete mixture containing all excipients, is processed through a conventional roller compactor to form ribbons, which are thereafter screened down to granules which are finally mixed with other excipients, like glidants, lubricants and antiadherents.

### BRIEF DESCRIPTION OF THE DRAWINGS

FIG. 1 is a flow diagram illustrating one embodiment of the direct compression manufacturing process to prepare a composition of to the present invention.

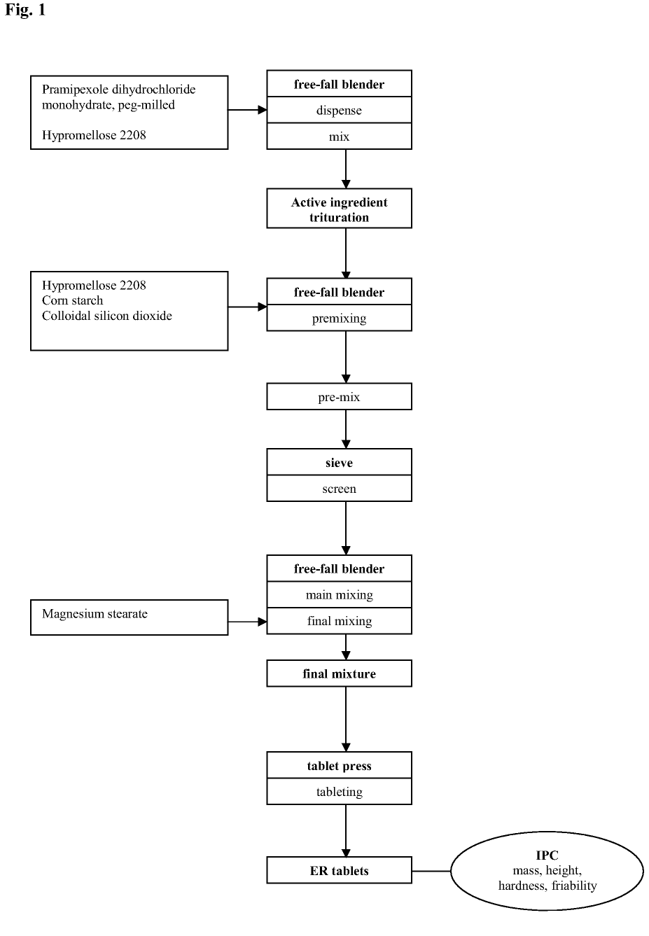

Process step (1) is directed to the active ingredient trituration, i.e. in the present case a salt of pramipexole, pramipexole dihydrochloride monohydrate, in peg-milled quality, is preblended with a portion of the polymer, in this case hydroxypropyl methylcellulose, in a commonly known mixer. In the flow chart a Turbula free-fall mixer or blender is used. The mixing time is several minutes, in the present case preferably 10 min.

In process step (2) according to the flow chart a premixing is performed, wherein the active ingredient trituration and the main portion of the water swelling polymer(s) and excipients are premixed for several minutes to obtain a pre-mix. In the present case the main portion of hydroxypropyl methylcellulose (hypromellose), corn starch, optionally carbomer (f.e. carbomer 941) and colloidal silicon dioxide are premixed for 5 min. in the above-mentioned Turbula mixer or blender.

According to the following process step (3) a dry screening may optionally take place. The pre-mixture may be manually screened through a screen, for example a 0.8 mm mesh size screen, in order to segregate cohesive particles and to improve content uniformity. The colloidal silicon dioxide optionally may be added after the sieving step.

In the subsequent process step (4) the main mixing step is performed according to which the components are mixed for several minutes, preferably 5 min. in the Turbula mixer after screening. Optionally further excipients may be added at this time, in the flow chart the component magnesium stearate is added to the main mixture, and further mixing for several minutes, e.g. 3 min., in the Turbula mixer is performed (final mixing) to obtain the final mixture.

Process step (5) of the process according to the present invention is the tableting. The final mixture is compressed on a suitable tablet press to produce, for example, oblong shaped matrix tablets (ER tablets=extended release tablets). In order to control and maintain the required quality the obtained matrix tablets are subjected to the following in-process controls: tablet mass, hardness, tablet height and friability.

The obtained pramipexole extended release tablets of the present invention may then be filled, for example, into High Density Polyethylene (HDPE) bottles. The bottles are closed tightly with screw caps and appropriately labelled, whereby all packaging and labelling activities are performed according to cGMP regulations. Alternatively, a blister type packaging can be used, e.g. using aluminium/aluminium foil blisters.

The present invention is further directed to the use of the extended release tablet formulation according to the present invention for preparing a medical composition for the treatment of any of the following diseases: Bipolar Disorder, Fibromyalgia, Restless Legs Syndrom, Parkinson Disease, in particular idiopathic Parkinson Disease, more particular idiopathic Parkinson Disease in an advanced stage, Dyskinesias and the like.

Bipolar Disorder is a manic-depressive disease, in that manic-stages, depressive stages and mixed stages may occur. The disease is characterised of unusual shifts in a person's mood, energy, and ability to function. Different from the normal ups and downs that everyone goes through, the symptoms of Bipolar Disorder are severe. They can result in damaged relationships, poor job or school performance, and even suicide. Scientifically one distinguishes between Bipolar I Disorder, Bipolar II Disorder, Cyclothymia and Bipolar Disorders Not Otherwise Specified. In Bipolar I Disorder full-fledged manic and major depressive episodes alternate. Among the criteria for Bipolar I Disorder are: single manic episodes, most recent episode hypomanic, most recent episode manic, moist recent episode mixed, most recent episode depressed, most recent episode unspecified. Bipolar I disorder commonly begins with depression and is characterized by at least one manic or excited period during its course. The depressive phase can be an immediate prelude or aftermath of mania, or depression and mania can be separated by months or years.

Bipolar II Disorder is characterised by recurrent major depressive episodes with hypomanic episodes. Cyclpthymidc disorder is a chronic, fluctuating mood disturbance which involves periods of hypomanic symptoms, and periods of depressive symptoms.

In Bipolar II Disorder usually depressive episodes alternate with hypomanias (relatively mild, nonpsychotic periods of usually less than 1 week). During the hypomanic period, mood brightens, the need for sleep decreases, and psychomotor activity accelerates beyond the patient's usual level. Often, the switch is induced by circadian factors (eg, going to bed depressed and waking early in the morning in a hypomanic state). Hypersomnia and overeating are characteristic and may recur seasonally (eg, in autumn or winter); insomnia and poor appetite occur during the depressive phase. For some persons, hypomanic periods are adaptive because they are associated with high energy, confidence, and supernormal social functioning. Many patients who experience pleasant elevation of mood, usually at the end of a depression, do not report it unless specifically questioned. Skillful questioning may reveal morbid signs, such as excesses in spending, impulsive sexual escapades, and stimulant drug abuse. Such information is more likely to be provided by relatives.

Patients with major depressive episodes and a family history of Bipolar Disorders (unofficially called Bipolar III Disorder) often exhibit subtle hypomanic tendencies; their temperament is termed hyperthymic (ie, driven, ambitious, and achievement-oriented).

Fibromyalgia is an increasingly recognized chronic pain illness characterized by widespread musculoskeletal aches, pain and stiffness, soft tissue tenderness, general fatigue and sleep disturbances. The most common sites of pain include the neck, back, shoulders, pelvic girdle and hands, but any body part can be involved. Fibromyalgia patients experience a range of symptoms of varying intensities that wax and wane over time.

The disease is characterized by the presence of multiple tender points and a constellation of symptoms. Patients have widespread pain over all parts of the body which often seems to arise in the muscles. The pain is profound, widespread and chronic. The pain is described as deep muscular aching, throbbing, twitching, stabbing and shooting pain. Neurological complaints such as numbness, tingling and burning are often present and add to the discomfort of the patient. The severity of the pain and stiffness is often worse in the morning. Aggravating factors that affect pain include cold/humid weather, non-restorative sleep, physical and mental fatigue, excessive physical activity, physical inactivity, anxiety and stress. Additionally to pain, patients commonly complain of fatigue in form of an all-encompassing exhaustion that interferes with even the simplest daily activities. Within the spectrum of symptoms are a decreased sense of energy, disturbances of sleep, problems with memory and concentration and varying degrees of anxiety and depression. Furthermore, certain other medical conditions are commonly associated with fibromyalgia, such as: tension headaches, migraine, irritable bowel syndrome, overactive bladder, pelvic pain, premenstrual tension syndrome, cold intolerance, skin sensitivities and rashes, dry eyes and mouth, anxiety, depression, ringing in the ears, dizziness, vision problems, Raynaud's Syndrome, neurological symptoms, impaired coordination and restless leg syndrome. Patients with established rheumatoid arthritis, lupus (SLE) and Sjogren's syndrome often develop fibromyalgia during the course of their disease.

Restless Leg Syndrome, also known as RLS, anxietas tibiarum, Syndrom Wittmaack-Ekbom-Syndrom, often realised as paresthesias (abnormal sensations) or dysesthesias (unpleasant abnormal sensations), is a neurological disorder which manifests itself chiefly as sensory disorders of the legs such as tingling, dragging, tearing, itching, burning, cramp or pain and in those affected triggers an irresistible compulsion to move. These sensations usually occur deep inside the leg, between the knee and ankle; more rarely, they occur in the feet, thighs, arms, and hands. Although the sensations can occur on just one side of the body, they most often affect both sides.

Frequently these disorders occur when the affected person is resting. Particularly at night, during sleep, these sensory disorders and the consequent compulsive movements lead to restlessness and sleep disorders. As a result, most people with RLS have difficulty falling asleep and staying asleep. Left untreated, the condition causes exhaustion and daytime fatigue. Many people with RLS report that their job, personal relations, and activities of daily living are strongly affected as a result of their exhaustion. They are often unable to concentrate, have impaired memory, or fail to accomplish daily tasks.

The symptoms of RLS vary in severity and duration from person to person. Mild RLS occurs episodically, with only mild disruption of sleep onset, and causes little distress. In moderately severe cases, symptoms occur only once or twice a week but result in significant delay of sleep onset, with some disruption of daytime function. In severe cases of RLS, the symptoms occur more than twice a week and result in burdensome interruption of sleep and impairment of daytime function.

The disease may begin at any time in life. Elderly people are more often affected than the younger. Usually, the disease is a chronic disease, which starts in a mild form, but usually the symptoms amplify over time.

The disease may be associated with or patients may develop further conditions, f.e. patients also may suffer from periodic limb movement disorder (PLMD). PLMD is characterized by involuntary leg twitching or jerking movements during sleep that typically occur every 10 to 60 seconds, sometimes throughout the night. The symptoms cause repeated awakening and severely disrupted sleep. Unlike RLS, the movements caused by PLMD are involuntary, meaning the patient has no control over them. Although many patients with RLS also develop PLMD, most people with PLMD do not experience RLS.

The formulation should be suited to treat RLS in children as well.

Parkinson's disease (PD) is considered to be a motor system disorder and accordingly, advanced stage in idiopathic Parkinson's disease is accompanied by motor dysfunction. The most frequent symptoms of PD are tremor, rigidity/akinesia, loss of dexterity, handwriting disturbances, gait disturbances, bradykinesia, postural instability, difficulty in swallowing and chewing, difficulties in speaking, urinary problems, constipation and/or other. Motor fluctuations may develop with the progression of the disease. Such changes are often referred to as late (motor)-complications of PD. Such late motor fluctuations and dyskinesia complications may have idiopathic origin as well as they may be caused by long-term dopaminergic treatment, f.e. with L-DOPA. In the progression of treatment with dopaminergic drugs side effects typically may increase over time, and the disease often manifests an “on-off” syndrome in advanced patients in which the drug simply doesn't work for unpredictable durations. In such stage periods with rapid fluctuations between uncontrolled movements and normal movement may occur, usually occurring after long-term use of L-DOPA. Advanced patients often have a “off”-time of more than 2 hours, more often more than 3 or even more than 4 hours a day.

The present invention is also interesting for to treat patients suffering from Parkinson's disease with dementia. In some instances of such patients, Magnetic Resonance Imaging (MIR), T1-weighted images or Computed Tomography (CT) Imaging reveal lesions in the cerebral white matter. They are not seen in parkinsonians without dementia.

A more systematic approach to define the stage of the Parkinson's disease are the modified Hoehn and Yahr scale or the Unified Parkinson Disease Rating Scale (UPDRS).

It may be considered that patients with a score of at least 2 to 3, preferably 3, more preferably 4 according the modified Hoehn and Yahr system are in an advanced stage of Parkinson's disease in the sense of the present invention. In this five stage disability scale stage one means least severe and stage five means most severe.

Stage One symptoms are signs and symptoms on one side only, symptoms mild, symptoms inconvenient but not disabling, usually presents with tremor of one limb, friends have noticed changes in posture, locomotion and facial expression.

Stage Two symptoms are symptoms are bilateral, minimal disability, posture and gait affected.

Stage Three symptoms are significant slowing of body movements, early impairment of equilibrium on walking or standing, generalized dysfunction that is moderately severe.

Stage Four symptoms are severe symptoms, can still walk to a limited extent, rigidity and bradykinesia, no longer able to live alone, tremor may be less than earlier stages.

Stage Five symptoms are cachectic stage, invalidism complete, cannot stand or walk, requires constant nursing care.

The Unified Parkinson Disease Rating Scale is a rating tool to follow the longitudinal course of Parkinson's Disease. It is made up of the following sections: 1) mentation, behavior, and mood, 2) activities of daily living and 3) motor. How to transfer this systematic to the severity of the disease can be taken from prior art. This system also may be used to define advanced stages of Parkinson's disease according to the present invention. In one embodiment, the formulation of the present invention can be used to treat patients with Parkinson's disease where depressed mood is the most cumbersome symptom. On the other hand the formulation is useful to treat motor symptoms of Parkinson's Disease. On the other hand the formulation is useful to treat motor symptoms of Parkinson's Disease.

It will be appreciated that it is up to the physician which kind of patients suffering from the disease he wants to treat with the active ingredient pramipexole, pramipexole dihydrochloride or another salt thereof respectively. According to the age of the elected patient, an adjustment of the dosage in the formulation of the invention will be necessary, in particular if children are to be treated.

The formulation according to the presenting invention and its advantages can be summarised as:
an oral modified or sustained or extended release composition comprising an active ingredient selected from pramipexole and its pharmaceutically acceptable salts, derivatives, solvates, and isomers, —in particular pramipexole dihydrochloride monohydrate—adapted for once daily administration.
an oral modified or sustained or extended release composition comprising an active ingredient selected from pramipexole and its pharmaceutically acceptable salts, derivatives, solvates, and isomers, wherein the active ingredient is released in vitro over a period of at least 4 hours, preferably 8 hours, and wherein the release profile is substantially independent of the pH of the dissolution medium at least in a certain pH-range, preferably in a pH-range of 4.5 to 7.5, with any or all of the following preferred characteristics:
   about 10 to about 45% of the active ingredient comprised in the composition is released after 3 hours, and wherein the pH of the dissolution medium is selected from about 1.2 to about 6.8, preferably from about 20 to about 65% of the active ingredient comprised in the composition is released after 6 hours, and wherein the pH of the dissolution medium is selected from about 1.2 to about 6.8.
   the initial hour of release is not more than about 25% of the active ingredient comprised in the composition is released.
   the amount of active ingredient released after 6 hours is ranging from about 100 to about 350 microgramm, or from about 150 to about 300 microgramm, calculated as pramipexole free base.
an oral modified or sustained or extended release composition comprising an active ingredient selected from pramipexole and its pharmaceutically acceptable salts, derivatives, solvates, and isomers, wherein the active ingredient is released in vitro over a period of at least 8 hours, and wherein the release profile is adapted to achieve a time to peak plasma concentrations (tₘₐₓ) of pramipexole of at least about 2.5 hours after administration to a human in the fasted state, with the following preferred characteristics:
   the mean time to peak plasma concentrations (tₘₐₓ) of pramipexole is at least 1 h longer than the mean time to peak plasma concentrations (tₘₐₓ) achieved by the administration of an oral immediate release dosage form comprising pramipexole dihydrochloride monohydrate.
an oral modified or sustained or extended release composition comprising an active ingredient selected from pramipexole and its pharmaceutically acceptable salts, derivatives, solvates, and isomers, wherein the active ingredient is released in vitro over a period of at least 4 hours, preferably 8 hours, and wherein the released amount of active ingredient after 4 hours at pH 6.8, when determined at a basket rotation speed of 100 rpm, is not more than about 80% of the released amount of active ingredient when determined at a basket rotation speed of 100 rpm after 4 hours at pH 6.8, with the following preferred characteristics:
   the released amount of active ingredient after 4 hours at pH 6.8, when determined at a basket rotation speed of 100 rpm, is not more than about 90% of the released amount of active ingredient when determined at a basket rotation speed of 100 rpm after 4 hours at pH 6.8.
an oral modified or sustained or extended release composition comprising an active ingredient selected from pramipexole and its pharmaceutically acceptable salts, derivatives, solvates, and isomers, wherein the release profile of the active ingredient is adapted to wherein the release profile is adapted to achieve average pramipexole plasma concentrations (C_{avg}) over a 24 h period which does not differ by more than 25% from the C_{avg} upon administration of an immediate release formulation at steady state after applying the same total daily dose of the immediate release formulation wherein the thrice daily administration is conducted at a time interval of about 6 hours between the first and the second administration and between the second and the third administration.
the dose of active ingredient is of about 0.5 to 1 mg, calculated as pramipexole free base.
an oral modified or sustained or extended release composition comprising an active ingredient selected from pramipexole and its pharmaceutically acceptable salts, derivatives, solvates, and isomers, wherein the release profile of the active ingredient is adapted to achieve a peak-through fluctuation (PTF) of pramipexole plasma concentrations upon once daily administration after reaching steady state of less than PTF after applying one third of the dose as immediate release formulation thrice daily wherein the thrice daily administration is conducted at a time interval of about 6 hours between the first and the second administration and between the second and the third administration.
   wherein the PTF of pramipexole plasma concentrations upon once daily administration after reaching steady state is preferably less than about 0.9.
   the peak plasma concentration of pramipexole (Cₘₐₓ) upon administration in the fasted state after reaching steady state is similar or lower than the peak plasma concentration of pramipexole (Cₘₐₓ) upon administration of an immediate release formulation at the same daily dose of the same active ingredient in the fasted state after reaching steady state.
   the trough plasma concentration of pramipexole (Cₘᵢₙ) upon administration in the fasted state after reaching steady state is higher than the trough plasma concentration of pramipexole (Cₘᵢₙ) upon administration of an immediate release formulation comprising a third of the dose of the same active ingredient in the fasted state after reaching steady state.
an modified or sustained or extended release formulation of Pramipexole, preferably a salt thereof, preferably the dihydrochloride, with the following preferred characteristics:
   the sustained release excipients is selected from the group of polymers, lipids and waxes, preferably a neutral or anionic polymer.
   the release-sustaining excipient and the active ingredient are both dispersed within one compartment.
   preferably it comprises at least one active ingredient-containing compartment and at least one release-sustaining compartment, said active ingredient-containing compartment being different from said release-sustaining compartment, preferably such that the release-sustaining compartment forms a layer on the active ingredient-containing compartment.
   preferably the release-sustaining compartment is poorly soluble in aqueous media at 37° C.
   it may comprise a plurality of active ingredient-containing compartments and a plurality of release-sustaining compartments, and wherein each active ingredient-containing compartment is associated with at least one release-sustaining compartment.
an oral modified or sustained or extended release composition comprising an active ingredient selected from pramipexole and its pharmaceutically acceptable salts, derivatives, solvates, and isomers, wherein the release profile of the active ingredient is substantially independent of the gastric residence time of the composition, preferably
   with a release profile of the active ingredient as determined by in vitro dissolution testing according to United States Pharmacopeia (USP) 28 using the basket apparatus (apparatus 1) set at a basket rotation speed of 100 rpm and using a dissolution medium of pH 1.2 that does not differ by more than about 20% from the release profile of the same composition using the same model and testing conditions except that the pH of the dissolution medium is 6.8.
   being designed as a solid dosage form adapted to disintegrate in gastric fluid at body temperature within less than about 30 minutes into active-ingredient-containing extended release particles having an average diameter of less than about 3.5 mm.
   with the particles that release the active ingredient over a period of at least 6 hours.

The formulation according to the invention may comprise any of these characteristics alone or in combination with other such characteristics. In particular it is for the treatment of any of the aforementioned indications.

The invention described will now be illustrated by the Examples which follow various other embodiments and will become apparent to the skilled person from the present specification. However, it is expressly pointed out that the Examples and description are intended solely as an illustration and should not be regarded as restricting the invention.

### EXAMPLES

### Example 1

### Preparation of Extended Release Composition

Extended release pellets were prepared, comprising pramipexole dihydrochloride monohydrate (0.91 wt.-%), microcrystalline cellulose (90.12 wt.-%), hydroxypropylcellulose (0.18 wt.-%), talc (0.79 wt.-%), ethylcellulose (6.40 wt.-%), and polyethylene glycol 6000 (1.6 wt.-%), by a two-step coating process starting from microcrystalline cellulose core pellets. In the first step, the core pellets were coated with an aqueous solution of the active ingredient and hydroxypropylcellulose as binder. In the second step, an organic solution of ethylcellulose and the plasticiser werde applied. The pellets were filled in two-piece HPMC hard capsules (size 3); the amount filled per capsule was calculated to yield a strength of 0.75 mg of active ingredient.

### Example 2

### Dissolution Testing of Extended Release Composition

Dissolution testing of the hard capsule composition prepared according to example 1 was conducted according to United States Pharmacopeia (USP) 28, chapter 711, using the same conditions and settings except for the composition and pH of the dissolution medium, which was varied between pH 1.3 and 7.3 (pH 1.3, 4.5, 6.8, and 7.3). Samples were taken after 1, 3, 6, 9, 12, 18 and 24 hours. In result, the average amount of drug released after 6 h was about 35%, after 12 h about 55%, and after 24 h about 70% of the incorporated dose. At no point of time, the difference in the released amount of drug between any of the dissolution profiles was more than 20% of the incorporated dose. Comparing the dissolution profiles at pH 4.5 and 6.3, there was no point of time at which there was a difference in the released amount of drug of more than 10% of the incorporated dose.

### Example 3

### Pharmacokinetics of Extended Release Composition

The hard capsule composition of example 1 was tested in 10 human volunteers for its pharmacokinetic properties at a regimen of multiple once daily dosing and compared to a commercially available tablet of pramipexole dihydrochloride monohydrate but having immediate release characteristics. The tablet was administered using a regimen of three dosings per day. In result, the hard capsule formulation achieved a mean time to maximum plasma concentrations of pramipexole (tₘₐₓ) of about 4.5 h. The mean average plasma concentration of pramipexole after reaching steady state was 0.47 ng/ml. Significantly, the fluctuation index as defined herein-above was substantially less than 100%, having a mean value of 57%. In contrast, the immediate release tablet exhibited a mean tₘₐₓ of only about 2 h, and a mean fluctuation index of 104%.

### Example 4

### Preparation of Extended Release Composition

Similar to example 1, extended release pellets were prepared and filled into HPMC hard capsules size 3. The dose of pramipexole dihydrochloride monohydrate per capsule was 0.75 mg. Relative to the weight of the pellets, they contained pramipexole dihydrochloride monohydrate (0.94 wt.-%), microcrystalline cellulose (92.40 wt.-%), hydroxypropylcellulose (0.19 wt.-%), talc (0.62 wt.-%), ethylcellulose (4.68 wt.-%), and polyethylene glycol 6000 (1.17 wt.-%). The same two-step coating process was used to obtain microcrystalline cellulose core pellets having a first coating comprising the active ingredient and an outer coating providing substantially pH-independent extended release characteristics.

### Example 5

### Dissolution Testing of Extended Release Composition

Dissolution testing of the hard capsule composition prepared according to example 4 was conducted as described before, using the same conditions and settings except for the pH of the dissolution medium, which was varied within the physiological range. Samples were taken after 1, 3, 6, 9, 12, 18 and 24 hours. In result, the average amount of drug released after 1 h was about 7%, after 3 h about 25%, after 9 h about 55%, after 12 h about 60%, and after 24 h about 75% of the incorporated dose. At no point of time, the difference in the released amount of drug between any of the dissolution profiles was more than 10% of the incorporated dose.

### Example 6

### Pharmacokinetics of Extended Release Composition

Similar to example 3, the hard capsule composition of example 4 was tested in 10 human volunteers for its pharmacokinetic properties at a regimen of multiple once daily dosing and compared to a commercially available tablet of pramipexole dihydrochloride monohydrate but having immediate release characteristics. The tablet was administered using a regimen of three dosings per day. In result, the hard capsule formulation achieved a mean time to maximum plasma concentrations of pramipexole (tₘₐₓ) of about 6.2 h. The mean average plasma concentration of pramipexole in after reaching steady state was 0.53 ng/ml. Again, the fluctuation index as defined herein-above was very small, having a mean value of 0.6.

1. An oral extended release composition comprising an active ingredient selected from pramipexole and its pharmaceutically acceptable salts, derivatives, solvates, and isomers, wherein the active ingredient is released in vitro over a period of at least 4 hours and wherein the release profile is adapted to achieve average pramipexole plasma concentrations (C_{avg}) over the release period which does not differ by more than 25% from the C_{avg} achieved at steady state upon administration of a thrice daily immediate release formulation of pramipexole at the same total daily dose of pramipexole as is administered using the extended release composition, wherein the thrice daily administration of the immediate release formulation is conducted at time intervals of about 6 hours between the first and the second administration and between the second and the third administration.

2. An oral extended release composition comprising an active ingredient selected from pramipexole and its pharmaceutically acceptable salts, derivatives, solvates, and isomers, wherein the active ingredient is released in vitro over a period of at least 4 hours and wherein the release profile is adapted to achieve a time to peak plasma concentrations (tₘₐₓ) of pramipexole of at least about 2.5 hours after administration to a human in the fasted state.

3. An oral sustained release composition comprising an active ingredient selected from pramipexole and its pharmaceutically acceptable salts, derivatives, solvates, and isomers, wherein the active ingredient is released in vitro over a period of at least 4 hours and wherein the released amount of active ingredient after 4 hours at pH 6.8, when determined at a basket rotation speed of 100 rpm, is not more than about 80% of the released amount of active ingredient when determined at a basket rotation speed of 100 rpm after 4 hours at pH 6.8.

4. An oral extended release composition comprising an active ingredient selected from pramipexole and its pharmaceutically acceptable salts, derivatives, solvates, and isomers, wherein the release profile of the active ingredient is adapted to achieve a peak-trough fluctuation (PTF) of less than that obtained after reaching steady state conditions with an immediate release formulation of pramipexole given thrice a day.

5. A composition according to any one of claims 1 to 4 having a substantially pH-independent release characteristic at least in the pH-range of 3.0 to 8.

6. A composition according to any one of claims 1 to 4 having a substantially pH-independent release characteristic in the pH-range of between 1 and below 8.

7. A composition according to any one of claims 1 to 4 being adapted for once daily administration.

8. A composition according to any one of claims 1 to 4 which provides a constant plasma level of the active ingredient over the whole gastrointestinal tract including colon.

9. A composition according to any one of claims 1 to 4 wherein the release profile of the active ingredient is substantially independent of the gastric residence time of the composition.

10. A composition according to any one of claims 1 to 4 in the form of a tablet that comprises pramipexole or a pharmaceutically acceptable salt thereof in a matrix comprising at least one water swelling polymer other than pregelatinized starch.

11. A composition according to any one of claims 1 to 4 in the form of a tablet having a non-functional coating.

12. A composition according to any one of claims 1 or 4, wherein the immediate release formulation is a tablet which comprises as inactive ingredients mannitol, corn starch, colloidal silicon dioxide, povidone, and magnesium stearate and as active ingredient pramipexole dihydrochloride monohydrate in an amount of either 0.125 mg or 0.25 mg or 0.5 mg or 1.0 mg or 1.5 mg or optionally more.

13. (canceled)

14. A method for treating Parkinson's disease in a patient, comprising administering to said patient a therapeutically effective amount of a composition according to any one of claims 1 to 4.

15. A method for treating RLS in a patient, comprising administering to said patient a therapeutically effective amount of a composition according to any one of claims 1 to 4.

16. A method for treating Bipolar Disorder, Fibromyalgia or Dyskinesias in a patient, comprising administering to said patient a therapeutically effective amount of a composition according to any one of claims 1 to 4.

17. A method according to claim wherein the composition is administered once daily.
